(19) United States
(12) Patent Application Publication
(10) Pub. No.: US 2022/0241603 A1
(43) Pub. Date: Aug. 4, 2022

(54) Varying Parameters of Tumor Treating Fields (TTFields) Treatment to Overcome Treatment Resistance

(71) Applicant: Novocure GmbH, Root D4 (CH)

(72) Inventor: Reuven R. SHAMIR, Haifa (IL)

(73) Assignee: Novocure GmbH, Root D4 (CH)

(21) Appl. No.: 17/590,408

(22) Filed: Feb. 1, 2022

Related U.S. Application Data
(60) Provisional application No. 63/147,961, filed on Feb. 10, 2021.
(60) Provisional application No. 63/145,371, filed on Feb. 3, 2021.

(51) Int. Cl. A61N 1/40

(52) U.S. Cl. CPC A61N 1/40

(57) ABSTRACT

Treatment resistance to Tumor Treating Fields (TTFields) has been observed in some subjects after those subjects were treated with TTFields for extended periods of time. Treatment resistance can be ameliorated by varying the treatment parameters of the TTFields over time. This can be accomplished by applying TTFields to cancer cells during a course of treatment, and varying a set of parameters of the TTFields during the course of treatment. Examples of parameters that can be varied during the course of treatment include the time of day at which the TTFields are applied, the frequency of the TTFields, the waveform used to generate the TTFields, the envelope shape of the waveform used to generate the TTFields, and the direction-switching rate of the TTFields.

### CROSS REFERENCE TO RELATED APPLICATIONS

This application claims the benefit of U.S. Provisional Applications 63/145,371 (filed Feb. 3, 2021) and 63/147,961 (filed Feb. 10, 2021), each of which is incorporated herein by reference in its entirety.

### BACKGROUND

Tumor Treating Fields, or TTFields, are alternating electric fields within the intermediate frequency range (e.g., 100-500 kHz) that inhibit cancer cell growth. This non-invasive treatment targets solid tumors and is described in U.S. Pat. No. 7,565,205, which is incorporated herein by reference in its entirety. 200 kHz TTFields are FDA approved for the treatment of glioblastoma (GBM), and may be delivered, for example, via the prior art Optune™ system. Optune™ includes a field generator and two pairs of transducer arrays (i.e., electrode arrays) that are placed on the patient's shaved head. One pair of arrays (L/R) is positioned to the left and right of the tumor, and the other pair of arrays (A/P) is positioned anterior and posterior to the tumor. In the preclinical setting, TTFields can be applied in vitro using, for example, the prior art Inovitro™ TTFields lab bench system.

FIG. 1 depicts how a set of operating parameters for the prior art Optune™ system remains constant over time. More specifically, the transducer arrays are always positioned at the same location on the subject's body. The power to the system is turned on every day at roughly the same time (e.g., around 6 AM) and turned off at roughly the same time every day (e.g., around 9 PM). The operating frequency is always 200 kHz. The amplitude is always the maximum amplitude that can be achieved without overheating the transducer arrays. And the field generator always (a) applies a sinusoidal AC voltage between the L/R transducer arrays (or electrodes) for a duration of time that is always 1 second; then (b) applies a sinusoidal AC voltage between the A/P transducer arrays (or electrodes) for the same duration of time; then repeats that two-step sequence (a) and (b) for the duration of the treatment.

### SUMMARY OF THE INVENTION

One aspect of the invention is directed to a first method of inhibiting growth of cancer cells. The first method comprises applying an alternating electric field to the cancer cells during a course of treatment, and varying a set of parameters of the alternating electric field during the course of treatment.

In some instances of the first method, the varying comprises varying the time of day at which the alternating electric field is applied over a course of multiple days. In some instances of the first method, the varying comprises using different frequencies on different days. In some instances of the first method, the varying comprises using different waveforms at different times within a single day. In some instances of the first method, the varying comprises using different waveforms on different days. In some instances of the first method, the varying comprises using different envelope shapes at different times within a single day. In some instances of the first method, the varying comprises using different envelope shapes on different days. In some instances of the first method, the varying comprises using different direction-switching rates at different times within a single day. In some instances of the first method, the varying comprises using different direction-switching rates on different days. In some instances of the first method, the varying comprises using different electrode positions at different times within a single day.

Another aspect of the invention is directed to a second method of inhibiting growth of cancer cells in a subject's body. The second method comprises applying an alternating electric field to the cancer cells in the subject's body during a course of treatment. The second method also comprises varying a set of parameters of the alternating electric field during the course of treatment, wherein the set of parameters includes at least one of (a) a time of day at which the alternating electric field is applied, (b) a frequency of the alternating electric field, (c) a waveform used to generate the alternating electric field, (d) an envelope shape of the waveform used to generate the alternating electric field, and (e) a direction-switching rate of the alternating electric field.

In some instances of the second method, the varying comprises varying the time of day at which the alternating electric field is applied over a course of multiple days. In some instances of the second method, the varying comprises using different frequencies on different days. In some instances of the second method, the varying comprises using different waveforms. In some instances of the second method, the varying comprises using different envelope shapes. In some instances of the second method, the varying comprises using different direction-switching rates.

Another aspect of the invention is directed to a first apparatus for inhibiting growth of cancer cells in a subject's body. The first apparatus comprises a signal generator having first and second outputs that operate in alternation, wherein the following parameters of the first and second outputs are controllable: a frequency, a waveform, an envelope shape of the waveform, and a rate of alternation between the first output and the second output. The first apparatus also comprises a controller configured to send control signals to the signal generator that cause the signal generator to vary at least one of the frequency, the waveform, the envelope shape, and the rate of alternation between the first output and the second output.

Some embodiments of the first apparatus further comprise first and second electrodes electrically connected to the first output of the signal generator, and third and fourth electrodes electrically connected to the second output of the signal generator.

In some embodiments of the first apparatus, the controller is programmed to generate control signals that cause the signal generator to operate using different rates of alternation between the first output and the second output.

### BRIEF DESCRIPTION OF THE DRAWINGS

FIG. 1 is a timing diagram that depicts how the parameters of the prior art Optune™ system remain constant over time.

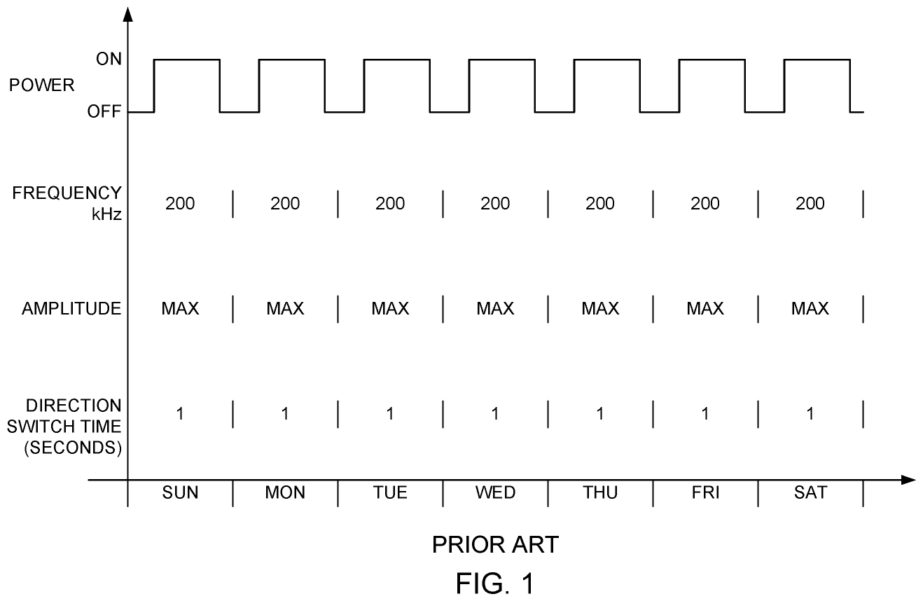

FIG. 2 is a block diagram of a system for driving a set of transducer arrays with AC voltage signals with non-constant operating parameters.

FIG. 3 is a timing diagram that depicts how the parameters of the TTFields may be varied over time in one embodiment of the invention.

Various embodiments are described in detail below with reference to the accompanying drawings, wherein like reference numerals represent like elements.

### DESCRIPTION OF THE PREFERRED EMBODIMENTS

Despite the clear benefit of TTFields therapy, treatment resistance is observed in some cases. Different patients at various times may respond differently to varying TTFields parameters, rather than fixed ones. The embodiments described herein therefore vary the treatment parameters over time. And this may be useful, for example, for patients that demonstrate treatment resistance. It may also be useful to prevent resistance from occurring, reducing the impact of resistance, and/or delaying the onset of resistance.

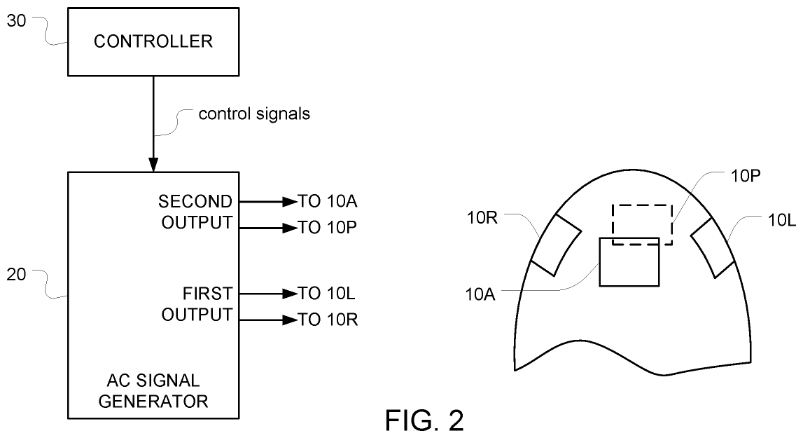

FIG. 2 is a block diagram of a system for driving a set of transducer arrays with AC voltage signals in which the operating parameters can be controlled. The system includes an AC signal generator 20 that is designed to generate first and second AC outputs at a frequency between 50 and 500 kHz. When the system is used to apply TTFields to a person's body (as shown in FIG. 2), the first AC output is applied across a first pair of electrodes 10L and 10R that are positioned to the left and right of the tumor; and the second AC output is applied across a second pair of electrodes 10A and 10P that are positioned anterior and posterior to a tumor. The AC signal generator 20 could also be used to apply TTFields to an in vitro culture (not shown) by applying the first AC output to electrodes positioned on the left and right walls of an Inovitro™ dish and applying the second AC output to electrodes positioned on the front and back walls of the Inovitro™ dish.

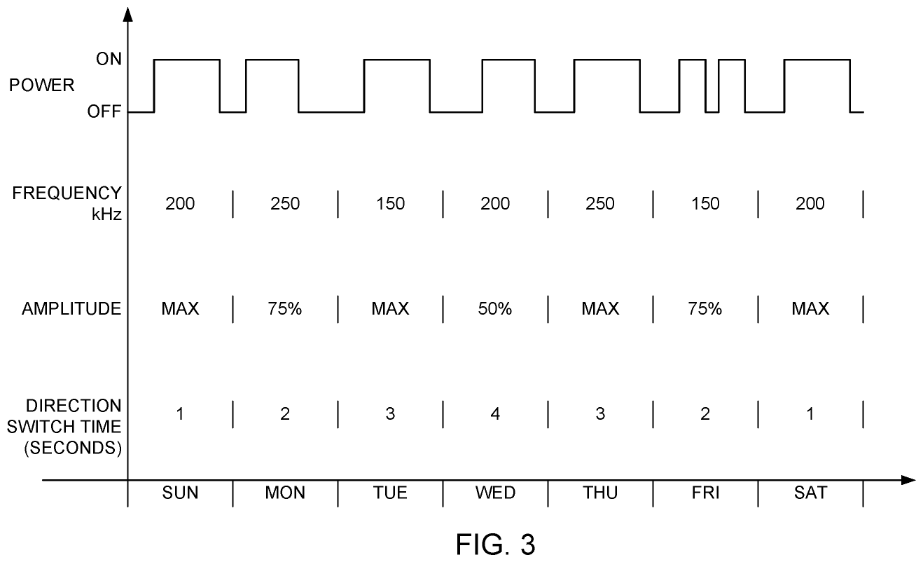

As in the prior art Optune™ and Inovitro™ systems, (a) the first AC output is applied to the L/R electrodes for an interval of time; (b) the second AC output is applied to the A/P electrodes for an interval of time; and the two-step sequence (a) and (b) is repeated for the duration of the treatment. But in contrast to Optune™, the operating parameters of the system can vary over time, as described in connection with FIG. 3.

FIG. 3 depicts four examples of approaches that may be used to vary the operating parameters in a TTFields system. The first row of FIG. 3 shows that instead of turning the system on at the same time every day and turning the system off at the same time every day, the operating time of the system may change. In the illustrated example, the system is on from 6 AM to 9 PM on Sunday, Tuesday, Thursday, and Saturday; the system is on from 3 AM to 3 PM on Monday; the system is on from 9 AM to 9 PM on Wednesday; and the system is on between 6 AM and noon and between 3 PM and 9 PM on Friday. The top row of FIG. 3 depicts only one example of how the power-on time may be varied, and a wide variety of alternative time patterns may be substituted for the illustrated example.

The second row of FIG. 3 shows that instead of having the system always operate at the same frequency (as in the prior art), the operating frequency of the system varies over time. In the illustrated example, the system operates at 200 kHz on Sunday, Wednesday, and Saturday; the system operates at 250 kHz on Monday and Thursday; and the system operates at 150 kHz on Tuesday and Friday. The second row of FIG. 3 depicts only one example of how the operating frequency may be varied over time, and a wide variety of alternative patterns may be substituted for the illustrated example. For example, instead of holding the operating frequency constant over the course of each day, the operating frequency may change within any given day. More specifically, on one day the operating frequency may be 200 kHz in the morning, 250 kHz in the afternoon, and 150 kHz in the evening; while on another day the operating frequency may be 150 kHz in the morning, 200 kHz in the afternoon, and 250 kHz in the evening.

The third row of FIG. 3 shows that instead of having the system always operate at the highest amplitude that will not cause the system to overheat, the system operates at a lower amplitude at certain times. In the illustrated example, the system operates at the highest amplitude on Sunday, Tuesday, Thursday, and Saturday, operates at 75% of that amplitude on Monday and Friday, and operates at 50% of the highest amplitude on Wednesday. The third row of FIG. 3 depicts only one example of how the amplitude may be varied, and a wide variety of alternative patterns may be substituted for the illustrated example. Here again, instead of holding the amplitude constant over the course of any given day, the amplitude may vary within any given day. More specifically, on one day the amplitude may be maximum before noon and 75% of that maximum in the afternoon; while on another day the amplitude may be maximum between noon and 4 PM, and 50% of maximum at all other times.

The fourth row of FIG. 3 shows that instead of having the system always switch the direction of the field every 1 second, the period of direction switching may vary at certain times. In the illustrated example, the switching rate is one second on Sunday and Saturday; two seconds on Monday and Friday; three seconds on Tuesday and Thursday; and four seconds on Wednesday. Once again, instead of holding the switching rate constant over the course of any given day, the switching rate may vary within any given day. For example, on one day the switching rate may be 1 second in the morning, 2 seconds in the afternoon, and 4 seconds in the evening; while on another day the switching rate may be 2 seconds in the morning, 1 second in the afternoon, and 3 seconds in the evening.

Other parameters that are not depicted in FIG. 3 may also be varied over time instead of holding those parameters constant as in the prior art. For example, instead of always applying a sinusoidal output waveform, the shape of the output waveform may be varied over time (e.g., by switching between a sinusoid, triangle wave, sawtooth, etc.) Another parameter that may be varied over time is the position of the electrodes on the subject's body.

Another parameter that may be varied over time is the directionality of the field. For example, instead of alternating the direction of the field between front/back and anterior/posterior, the direction of the field may be held constant during some days or portions of days. Alternatively or additionally, additional directions for the fields may be obtained by using additional sets of electrodes, and the directionality of the field may be varied.

Another parameter that may be varied over time is the shape of the envelope of the AC signal that is applied between given sets of electrodes during any given interval where TTFields are applied in a given direction (e.g., the 1 second intervals described above). For example, the envelope of the AC signal during a given interval may jump up to a maximum quickly and remain at that maximum over the course of the interval; the envelope of the AC signal during a given interval may ramp up to a maximum and then ramp back down from that maximum over the course of the interval; or the envelope of the AC signal during a given interval may start off at zero, jump up to a fixed value, then returned to zero during the course of the interval.

Another parameter that may be varied is the time that the electrodes remain on the subject's body before the electrodes are replaced. For example, instead of replacing the electrodes on a fixed schedule (e.g., every 10 days), the electrodes could be replaced on a variable schedule (e.g., by sometimes replacing the electrodes after 7 days, and sometimes replacing the electrodes after 12 days).

Another parameter that may be varied is the nature of skin care regimens that are used during the course of the TTFields treatment. For example, a first skin care regimen may be performed just prior to application of the electrodes to the subject's body on certain days; and a second skin care regimen may be performed just prior to application of the electrodes to the subject's body on other days.

Another parameter that may be varied is the nature of the hydrogel that is used during the course of the TTFields treatment. For example, a first type of hydrogel may be applied just prior to application of the electrodes to the subject's body on certain days; and a second type of hydrogel may be applied just prior to application of the electrodes to the subject's body on other days.

Another parameter that may be varied is the shape and configuration of the electrode arrays that are used to apply TTFields to a subject's body. For example, arrays of electrodes with one configuration may be used during certain time intervals (e.g., 7-12 day intervals); and arrays of electrodes with another configuration may be used during other time intervals (e.g., 7-12 day intervals). Another parameter that may be varied is the way that the electrodes are positioned against the subject's body. For example, during some intervals of time the electrodes may be held against the subject's body using an adhesive; and during other intervals of time the electrodes may be held against the subject's body by gravity (e.g., by incorporating one array of electrodes into a pad positioned beneath a supine or prone subject and incorporating another array of electrodes into a blanket positioned on top of the subject).

When additional treatment modalities (e.g., chemotherapy and/or radiation) are used in addition to TTFields therapy, additional parameters may be varied, including but not limited to varying the timing relationship between the TTFields therapy and the other treatment modalities. For example, in some instances the chemotherapy may be administered on the same day that the TTFields electrodes are replaced; while in other instances the chemotherapy may be administered two days after the TTFields electrodes are replaced.

Returning to FIG. 2, the AC signal generator 20 is configured to generate first and second AC outputs such that the first and second AC outputs have parameters that depend on a state of at least one control input.

A controller 30 continuously sends control signals to the at least one control input to achieve the desired output parameters, which are varied as described herein.

The details of the construction of the controller 30 and the nature of the control signals will depend on the design of the AC signal generator 20. In one example, the design of the AC signal generator 20 is similar to the AC signal generator described in U.S. Pat. No. 9,910,453, which is incorporated herein by reference in its entirety. This particular AC signal generator has two output channels (i.e., a first channel for L/R and a second channel for A/P). The instantaneous AC output voltage on either channel depends on the instantaneous output voltage of a DC-DC converter, and the output voltage of that DC-DC converter is controlled by writing a control word to a digital-to-analog converter (DAC). This AC signal generator can therefore be used to generate output signals that have the desired parameters that vary over time, as described herein.

The controller 30 controls the AC signal generator 20 by writing an appropriate sequence of control words to the DAC within the AC signal generator 20 at appropriate times, in order to cause the AC signal generator 20 to generate the desired output waveforms.

A wide variety of alternative designs for the AC signal generator 20 and the controller 30 can be substituted for the example provided above, as long as the controller 30 has the ability to control the AC signal generator 20. For example, if the AC signal generator is designed to respond to an analog control signal, the controller 30 must generate whatever sequence of analog control signals is needed to cause the AC signal generator 20 to output the desired waveforms. In this situation, the controller 30 could be implemented using a microprocessor or microcontroller that is programmed to write appropriate control words to a digital-to-analog converter, the output of which generates the analog control signals that causes the AC signal generator 20 to generate the desired waveforms. Alternatively, the controller 30 could be implemented using an analog circuit that automatically generates the appropriate sequence of control signals (which are then applied to the control input of the AC signal generator).

The frequency of the alternating electric fields could vary, for example, between 50 and 500 kHz, or between 50 kHz and 1 MHz. The direction of the alternating electric fields can be switched at rates, for example, between 1 ms and 360 seconds.

The electrical field may be capacitively coupled into the subject (in in vivo situations) or into a culture (in in vitro situations) by interposing a dielectric layer between the electrodes and the subject/culture. But in alternative embodiments, the electric field could be applied directly to the cells without capacitive coupling.

The methods described herein can be applied in the in vivo context by applying the alternating electric fields to a target region of a live subject's body, for both glioblastoma cells and other types of cancer cells. This may be accomplished, for example, by positioning electrodes on or below the subject's skin so that application of an AC voltage between selected subsets of those electrodes will impose the alternating electric fields in the target region of the subject's body.

For example, in situations where the relevant cells are located in the subject's lungs, one pair of electrodes could be positioned on the front and back of the subject's thorax, and a second pair of electrodes could be positioned on the right and left sides of the subject's thorax. In some embodiments, the electrodes are capacitively coupled to the subject's body (e.g., by using electrodes that include a conductive plate and also have a dielectric layer disposed between the conductive plate and the subject's body). But in alternative embodiments, the dielectric layer may be omitted, in which case the conductive plates would make direct contact with the subject's body. In another embodiment, electrodes could be inserted subcutaneously below a patient's skin. An AC voltage generator applies an AC voltage at a selected frequency between the right and left electrodes for a first period of time, which induces alternating electric fields where the most significant components of the field lines are parallel to the transverse axis of the subject's body. Then, the AC voltage generator applies an AC voltage at the same frequency (or a different frequency) between the front and back electrodes for a second period of time, which induces alternating electric fields where the most significant components of the field lines are parallel to the sagittal axis of the subject's body. This two step sequence is then repeated for the duration of the treatment.

Optionally, thermal sensors may be included at the electrodes, and the AC voltage generator can be configured to decrease the amplitude of the AC voltages that are applied to the electrodes if the sensed temperature at the electrodes gets too high. In some embodiments, one or more additional pairs of electrodes may be added and included in the sequence. In alternative embodiments, only a single pair of electrodes is used, in which case the direction of the field lines is not switched.

While the examples described above involve parameters that are varied in a deterministic manner, the variation of those parameters may be random or pseudorandom instead of deterministic. The variations of the parameters (whether random or deterministic) are preferably bounded within a specific range of parameters that ensure safety and that maximize likelihood of efficacy.

Using time-varying parameters instead of constant parameters may be useful for patients that demonstrate treatment resistance. It may also be useful for preventing resistance from occurring, reducing the impact of resistance, and/or delaying the onset of resistance.

To ensure efficacy, in-vitro or in-vivo data can be utilized. For example, cancer cells that demonstrate treatment resistance may be grown on a dish. Then, the treatment parameters may be varied in many ways to find a set of variations that reduces cancer cell growth the most. The cells may also be characterized into details (genes, histology, etc.) and optimal treatment-resistance alternation schemes may be developed per specific cancer cell portfolio. Moreover, with time and as the patient's specific imaging and clinical data is gathered, the treatment parameters may be changed or alternated, based on the individual patient's specific situation. Biomarkers and other inputs from sensors on the patient may also be used for that. Once enough patient population data is collected, this method can be extended to incorporate it as well. A best treatment alternation scheme may then be recommended based on multiple past patients' data. the response of patients of similar prognosis, health history, cell characteristics and etc. to treatment and treatment alternations may facilitate the effective recommendation for optimal treatment parameters at the right time. By using the methods and apparatuses described herein, the growth of cancer cells can be inhibited.

While the present invention has been disclosed with reference to certain embodiments, numerous modifications, alterations, and changes to the described embodiments are possible without departing from the sphere and scope of the present invention, as defined in the appended claims. Accordingly, it is intended that the present invention not be limited to the described embodiments, but that it has the full scope defined by the language of the following claims, and equivalents thereof.

What is claimed is:

1. A method of inhibiting growth of cancer cells, the method comprising:
applying an alternating electric field to the cancer cells during a course of treatment; and
varying a set of parameters of the alternating electric field during the course of treatment.

2. The method of claim 1, wherein the varying comprises varying the time of day at which the alternating electric field is applied over a course of multiple days.

3. The method of claim 1, wherein the varying comprises using different frequencies on different days.

4. The method of claim 1, wherein the varying comprises using different waveforms at different times within a single day.

5. The method of claim 1, wherein the varying comprises using different waveforms on different days.

6. The method of claim 1, wherein the varying comprises using different envelope shapes at different times within a single day.

7. The method of claim 1, wherein the varying comprises using different envelope shapes on different days.

8. The method of claim 1, wherein the varying comprises using different direction-switching rates at different times within a single day.

9. The method of claim 1, wherein the varying comprises using different direction-switching rates on different days.

10. The method of claim 1, wherein the varying comprises using different electrode positions at different times within a single day.

11. A method of inhibiting growth of cancer cells in a subject's body, the method comprising:
applying an alternating electric field to the cancer cells in the subject's body during a course of treatment; and
varying a set of parameters of the alternating electric field during the course of treatment, wherein the set of parameters includes at least one of (a) a time of day at which the alternating electric field is applied, (b) a frequency of the alternating electric field, (c) a waveform used to generate the alternating electric field, (d) an envelope shape of the waveform used to generate the alternating electric field, and (e) a direction-switching rate of the alternating electric field.

12. The method of claim 11, wherein the varying comprises varying the time of day at which the alternating electric field is applied over a course of multiple days.

13. The method of claim 11, wherein the varying comprises using different frequencies on different days.

14. The method of claim 11, wherein the varying comprises using different waveforms.

15. The method of claim 11, wherein the varying comprises using different envelope shapes.

16. The method of claim 11, wherein the varying comprises using different direction-switching rates.

17. An apparatus for inhibiting growth of cancer cells in a subject's body, the apparatus comprising:
a signal generator having first and second outputs that operate in alternation, wherein the following parameters of the first and second outputs are controllable: a frequency, a waveform, an envelope shape of the waveform, and a rate of alternation between the first output and the second output; and
a controller configured to send control signals to the signal generator that cause the signal generator to vary at least one of the frequency, the waveform, the envelope shape, and the rate of alternation between the first output and the second output.

18. The apparatus of claim 17, further comprising:
first and second electrodes electrically connected to the first output of the signal generator; and
third and fourth electrodes electrically connected to the second output of the signal generator.

19. The apparatus of claim 17, wherein the controller is programmed to generate control signals that cause the signal generator to operate using different rates of alternation between the first output and the second output.
